(19) United States
(12) Patent Application Publication
(10) Pub. No.: US 2011/0093211 A1
(43) Pub. Date: Apr. 21, 2011

(54) Method for Processing Protein Data

(75) Inventors: Andre' Xavier de Carvalho Negrão Valente, Cantanhede (PT); Yuan Gao, Richmond, VA (US); Gregory A. Buck, Richmond, VA (US); Seth Roberts, Richmond, VA (US)

(73) Assignees: BIOCANT-ASSOCIAÇÂO DE TRANSFERÉNCIA DE TECNOLOGIA; Virginia Commonwealth University Intellectual Property foundation

(21) Appl. No.: 12/997,441

(22) PCT Filed: Jun. 10, 2009
(86) PCT No.: PCT/EP09/04169

§ 371 Date: Dec. 10, 2010

Related U.S. Application Data
(60) Provisional application No. 61/131,929, filed on Jun. 13, 2008.

(30) Foreign Application Priority Data
Jun. 9, 2009 (PT) 104617

(51) Int. Cl. G06F 19/00; G01N 33/68

(52) U.S. Cl. 702/19

(57) ABSTRACT

A method of generating data indicating whether a set of proteins is a protein complex. The method comprises receiving as input experimental data indicating experimentally observed relationships, each experimentally observed relationship being between a first protein and zero or more second proteins and generating data indicating whether the set of proteins is a protein complex. The experimental data is processed to determine a first data value indicating a number of proteins having a relationship with one or more second proteins and a second data value indicating a number of proteins having a relationship with a selected protein.

The present invention relates to a method of generating data indicating whether a set of proteins is a protein complex. The invention also relates to a method of generating data indicating a set of protein complexes.

Proteins are vital components of living organisms. They have a crucial role as the main elements of cellular metabolic pathways. The “proteome” is the entire complement of proteins of an organism, and the term “proteomics” is used to describe the large-scale study of proteins, particularly with respect to their structures and functions.

Most proteins function in collaboration with other proteins. As well as playing a central role in many biological functions, the interactions between proteins are important for many diseases. For example, signals from the exterior of a cell may be mediated to the inside of that cell by protein-protein interactions of the signaling molecules. This process, called signal transduction, plays a fundamental role in many biological processes and in many diseases (e.g. cancer). It is hoped that comprehensive mapping of protein physical interactions will facilitate novel insights, regarding both fundamental cell biology processes and the pathology of diseases.

It is recognised that there are different types of protein-protein interaction. For example, proteins might interact for a long time to form part of a protein complex; or a protein may be carrying another protein (for example, from cytoplasm to nucleus or vice versa in the case of the nuclear pore importins); or a protein may interact briefly with another protein just to modify it (for example, a protein kinase will add a phosphate to a target protein).

A protein complex can be considered to be a group of two or more associated proteins formed by protein-protein interaction that is stable over time. Protein complexes are a form of quaternary structure. Many protein complexes have been identified, particularly in the model organism Saccharomyces cerevisiae, a yeast. The discovery of protein complexes is now performed genome wide; the elucidation of most protein complexes of the yeast is undergoing. Understanding the functional interactions of proteins is an important research focus in biochemistry and cell biology.

An important aim of proteomics is to identify which proteins interact; i.e. to identify a map of “protein-protein interactions” within a given cell. The collection of protein physical interactions present in a cell, termed the “interactome”, constitutes a cornerstone in the field of “Systems Biology”, being the most fundamental level at which it is possible to perform an integrated analysis of a cell rather than just an isolated study of individual components.

Various experimental methods have been adopted to identify protein-protein interactions and protein complexes, such as for example affinity purification and yeast two hybrid (Y2H). Affinity purification is considered as a low-throughput method (LTP) suited to identify protein complexes. An advantage of this method is that there can be real determination of protein partners quantitatively in vivo without prior knowledge of complex composition. It is also simple to execute and often provides high yield. Y2H, in contrast, is suited to explore the binary interactions in mass quantities and is considered as a high-throughput method (HTP). Each of the approaches has its own strengths and weaknesses, especially with regard to the sensitivity and specificity of the method. A high sensitivity means that many of the interactions that occur in reality are detected by the screen. A high specificity indicates that most of the interactions detected by the screen are also occurring in reality.

It is anticipated that the comprehensive mapping of protein physical interactions will facilitate the understanding of fundamental cell biology processes and the pathology of diseases. However, it is crucial to address existing problems. In particular, how to obtain reliable interaction data in a high-throughput setting. This is important as high-throughput methods allow for the mapping of entire protein physical interactions present in a cell, i.e. an interactome.

It is an object of embodiments of the present invention to obviate or mitigate one or more of the problems set out above.

According to a first aspect of the present invention, there is provided a method of generating data indicating whether a set of proteins is a protein complex, the method comprising: receiving as input experimental data indicating experimentally observed relationships, each experimentally observed relationship being between a first protein and zero or more second proteins; generating data indicating whether the set of proteins is a protein complex by processing said experimental data to determine: a first data value indicating a number of proteins having a relationship with one or more second proteins; and a second data value indicating a number of proteins having a relationship with a selected protein.

The term “protein complex” is used herein to include a group of two or more proteins formed by protein-protein interaction that is stable over a period of time, as can be appreciated by the skilled person.

The first aspect of the present invention is based upon the inventors' surprising realisation that processing data indicating first and second data values of the type set out above can provide information useful in identifying protein complexes.

In particular, the inventors have found that finding a ratio of the first and second data values and comparing the ratio to a predetermined threshold provides information usable in the identification of protein complexes. The method may therefore further comprise generating relationship data indicating a relationship between the first data value and the second data value, and the data indicating whether the set of proteins is a protein complex may be based upon the relationship data.

Some embodiments of the invention can therefore provide an improved method of analysing high-throughput interaction data to identify protein complexes using a computational algorithm. The inventors have applied the improved method to construct a new interactome for S cerevisiae, and demonstrated that it yields reliability typical of low-throughput experiments out of high-throughput data. Hence the method can be use to identify biologically important protein complexes, particularly those having a role in human disease.

In some embodiments data from a high throughput protein identification assay can be used to prepare an interactome.

The method of the first aspect of the invention may further comprise determining whether the relationship data satisfies a predetermined condition. The predetermined condition may be defined with reference to a threshold. Data indicating that the set of proteins is a protein complex may be generated if the predetermined condition is satisfied. Data indicating that the set of proteins is not a protein complex may be generated if the predetermined condition is not satisfied. Data indicating that the set of proteins is a protein complex may be generated if but only if the set of proteins is not a subset of another set of proteins which is a protein complex.

The experimental data may be any protein-protein interaction data. For example, the data may be derived from protein-protein interaction prediction experiments such as phylogenetic profiling; prediction of co-evolved protein pairs based on similar phylogenetic trees; identification of homologous interacting pairs; identification of structural patterns; or bayesian network modelling. The data may be derived from protein-protein interaction screening experiments using techniques such as ex vivo or in vivo methods including Bimolecular Fluorescence Complementation or the yeast two-hybrid screen; or in vitro methods including affinity purification (preferably TAP) or chemical crosslinking.

Preferably the experimental data is “pulldown” assay data in which proteins that interact with a selected protein are isolated using affinity purification techniques (preferably TAP) in which the selected protein is used as “bait”. Any such isolated protein is subsequently identified, typically using mass spectrometric analysis. Various different techniques can be used to derive pulldown assay data. It is important to point out that the method of the invention need not include the step of deriving the experimental data.

Preferably the experimental data is protein-protein interaction data of a eukaryotic cell. Such data may be derived from yeast (for example Saccharomyces cerevisiae or Schizosaccharomyces pombe). More preferably the data is derived from a mammalian cell, most preferably a human cell. The experimental data may be derived from many different types of human cell; preferably the human cell has a disease state, for example a cancerous human cell.

Data indicating the set of proteins may be stored. The first data value may indicate a number of proteins in the set, other than the selected protein, having a relationship with one or more second proteins, and the second data value may indicate a number of proteins in the set, having a relationship with the selected protein.

Each protein of the set of proteins may be selected in turn to be the selected protein. A plurality of first data values may be generated, one for each protein of the set of proteins. A plurality of second data values may be generated, one for each protein of the set of proteins.

Relationship data may be generated for each protein in the set of proteins based upon respective first and second data values. The set of proteins may be identified as a protein complex if but only if the relationship data for each protein in the set of proteins satisfies a predetermined condition.

The experimental data indicating experimentally observed relationships may comprise a plurality of relationships between a particular first protein and a respective zero or more second proteins. The method may further comprise determining a proportion of the plurality of relationships indicating that the particular first protein has a relationship with the selected protein.

At least one of the first and second data values may be modified based upon a number of first proteins in the experimental data having a relationship with the selected protein. The modifying may be based upon a number of first proteins in the experimental data having a relationship with one or more other proteins. Modifying the at least one of the first and second data values may use a discount value which is defined with reference to a probability of obtaining by chance a value of the second data value greater than or equal to said discount value.

The set of proteins may be defined with reference to one or more second proteins with which a first protein has a relationship.

According to a second aspect of the present invention there is provided a method of generating data indicating a set of protein complexes comprising: generating data indicating a set of sets of proteins; processing each set of proteins according to the method of the first aspect of the invention and generating data indicating a set of protein complexes based upon the processing.

In the second method of the invention, each set of proteins may be defined with reference to one or more second proteins with which a first protein has a relationship.

The method may further comprise generating data indicating a set of sets of proteins, each set of proteins comprising a pair of proteins. Each set of proteins comprising a pair of proteins may be processed using a method according to the first aspect of the invention. Data indicating a set of protein complexes may be generated based upon the processing. The set of sets of proteins may be generated to include each pair of proteins which may be defined with reference to proteins included in the experimental data.

The method may further comprise generating data indicating a merged set of sets of proteins, each set of proteins comprising all proteins included in a plurality of protein complexes indicated by the generated data. Each set of proteins in the merged set of sets of proteins may be processed using a method according to a first aspect of the invention. The data indicating the set of protein complexes may be modified based upon the processing. The merged set may be generated to include each pair of protein complexes indicated by the generated data.

The method may further comprise generating data indicating a further set of proteins comprising all proteins included in a selected one of the protein complexes indicated by the generated data and at least one further protein. The further set may be processed using a method according to a first aspect of the invention and the data indicating the set of protein complexes may be modified based upon the processing.

The method may further comprise repeatedly carrying out the processing of combining pairs of proteins and carrying out the processing of combining pairs of protein complexes until no further sets of proteins can be created using the processing which have not been processed.

The method may further comprise selecting first and second protein complexes indicated by the generated data. It may be determined whether a predetermined proportion of proteins of the first protein complex are also proteins of the second protein complex. It may be further determined whether the number of proteins in the first protein complex is greater than or equal to the number of proteins in the second protein complex, and the data indicating protein complexes may be modified to remove the second protein complex if both tests are satisfied.

The data indicating protein complexes may be processed to determine whether the proteins of a first protein complex form a subset of the proteins of a second protein complex. If the proteins of a first protein complex do form a subset of the proteins of a second protein complex, the generated data may be modified to remove the first protein complex.

The invention further provides a method of determining whether two protein complexes transiently interact. That is, a method is provided for generating data indicating whether two protein complexes form a transient protein complex. The method comprises receiving data defining two protein complexes; determining whether proteins of said two protein complexes satisfy a predetermined relationship; and generating data indicating whether said two protein complexes transiently interact based upon said determining.

Determining whether proteins included in said two protein complexes satisfy a predetermined relationship may comprise selecting a protein included in one of said two protein complexes, and processing experimental data based upon said selected protein to determine whether said two protein complexes transiently interact. The selected protein is preferably included in only one of said two protein complexes.

The experimental data may indicate a relationship between said selected protein and a plurality of other proteins. For example, the experimental data may indicate proteins pulled down when the selected protein is used as a bait.

The processing may determine whether said experimental data includes at least a first predetermined number of proteins included in said first protein complex and a second predetermined number of proteins included in said second protein complex. The first predetermined number of proteins may be half the number of proteins included in said first protein complex, and said second predetermined number of proteins may be half the number of proteins included in said second protein complex. That is, the processing may determine whether the experimental data indicates that at least 50% of proteins in each of the first and second protein complexes are pulled down when the selected protein is used as a bait.

Therefore, when data indicating a set of protein complexes has been generated, a set of predicted putative pair-wise transient interactions between these protein complexes represented by the generated data may be assembled, by submitting each pair of complexes to the less stringent test of partially appearing together in a single experimental assay.

From a functional perspective, transient interactions can usefully be considered as comprising two qualitatively distinct types, herein termed ‘wide-ranging’ and ‘restricted’. The ‘wide-ranging’ interaction is that associated with a protein/complex performing a standard function on many target proteins/complexes. An example of interactions of this type are those between a chaperone and its potentially hundreds of targets. The ‘restricted’ kind of transient interaction is the one that occurs when two proteins/complexes come together in a more delimited functional context, for example a kinase substrate transient interaction within a particular signaling pathway. Both kinds are of relevance, but due to their functionally distinct nature, they are best addressed separately, in particular so that, due to its pervasiveness, the wide-ranging kind does not occlude the restricted kind, as may be the case under the concept of hubs.

In an interactome map created using the methods described herein, attempts are made to screen out the wide-ranging type transient interactions by excluding predicted transient interactions of complexes involved in more than a specified cut-off number of predicted transient interactions (preferably, 8 interactions). A detailed description of both the permanent complex prediction algorithm and the transient interaction prediction algorithm, is given below.

The inventors have been concerned with the problem of how to structure interaction data in a meaningful form so as to be amenable and valuable for further biological research. From the point of view of the biological usefulness of the generated data, structuring of the interaction data in terms of permanent complexes and transient complexes is an improvement over techniques which treat all interactions equally, or consider only permanent protein complexes.

Being of lower affinity, as they are complex-complex interactions as well as protein-protein interactions, the predicted transient interactions are harder to discern; indeed there is currently little data on transient complex-complex interactions.

Nonetheless the reliability of the data derived from methods implementing aspects of the invention was assessed using a number of different tests, each of which are further described in the accompanying examples. Briefly, Semantic Distance tests show that for both the GO Biological Process and the GO Cellular Component annotations, the average Semantic Distance associated with this class of interactions is higher than the respective average for permanent complexes, while lower than the respective average for the class of wide-ranging interactions consistent with expectations. Examples of interactions between protein complexes predicted according to the second method of the invention are provided in the accompanying examples.

A further aspect of the invention provides computer programs comprising computer readable instructions controlling a computer to carry out a method as set out above. The computer program may be carried on a suitable carrier medium. Such a carrier medium may be a tangible carrier medium such as a hard drive, CD-ROM or floppy disk or alternatively an intangible carrier medium such as a communications signal.

A further aspect of the invention provides apparatus for generating data indicating whether a set of proteins is a protein complex. The apparatus comprises a memory storing processor readable instructions; and a processor configured to read and execute instructions stored in the program memory. The processor readable instructions comprise instructions controlling the processor to carry out a method as set out above.

The reliability of the data derived from the method of embodiments of the invention was assessed using a number of different tests, each of which are further described in the accompanying example. Briefly, the protein complexes predicted according to the method of the invention were compared to manually curated complexes from the MIPS database; they were assessed using Semantic Distance analysis; and they were assessed according to an “essentiality” test. Taken together, the results from such analysis demonstrated that method of embodiments of the invention allows large-scale prediction of complexes with a reliability typical of low-throughput experiments from experimental data. Examples of protein complexes predicted from the method of this aspect of the invention are provided in the accompanying examples.

Embodiments of the invention will now be described, by way of example, with reference to the accompanying drawings, in which:

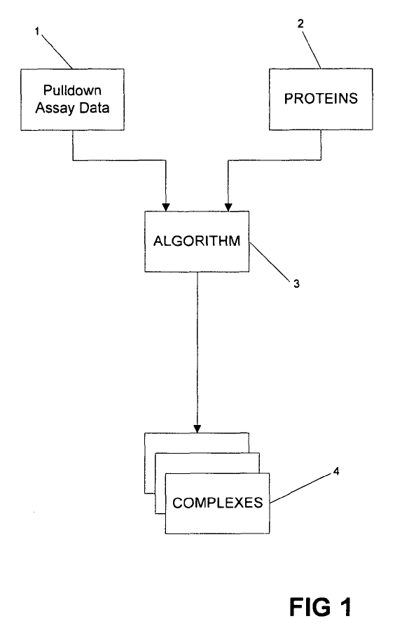

FIG. 1 is a schematic illustration of processing carried out in an embodiment of the present invention;

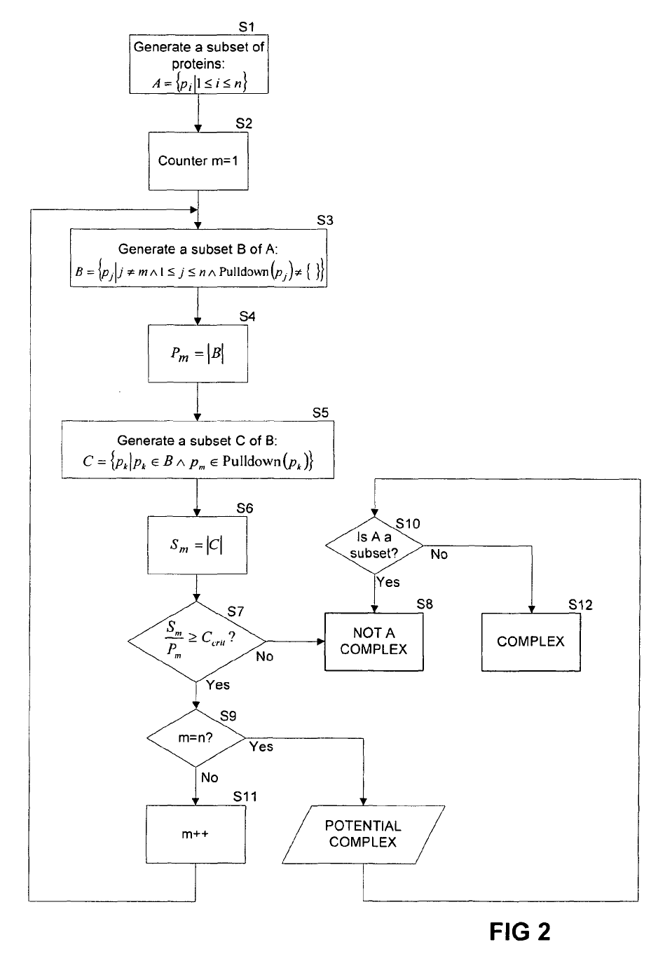

FIG. 2 is a flowchart showing processing to determine whether a criterion which is to be satisfied by protein complexes is satisfied;

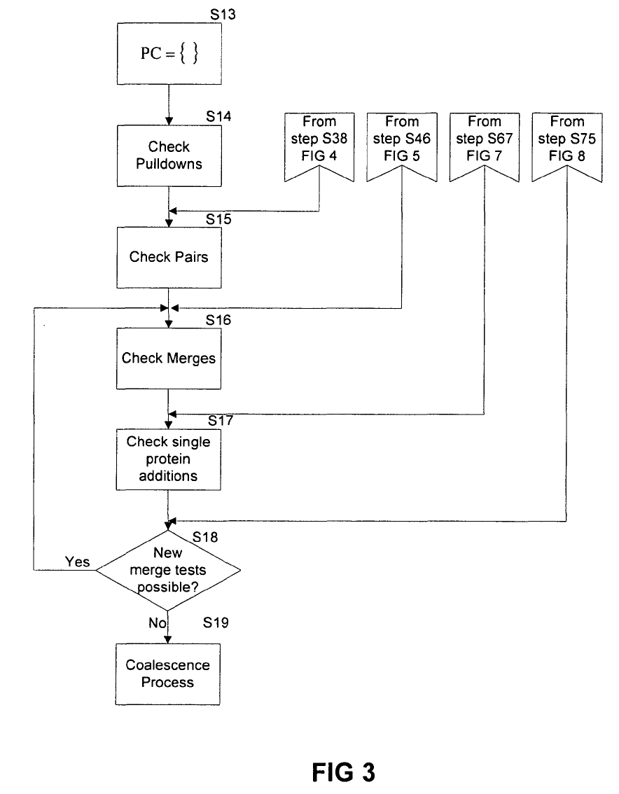

FIG. 3 is a flowchart showing, in overview, processing carried out to generate a set of protein complexes;

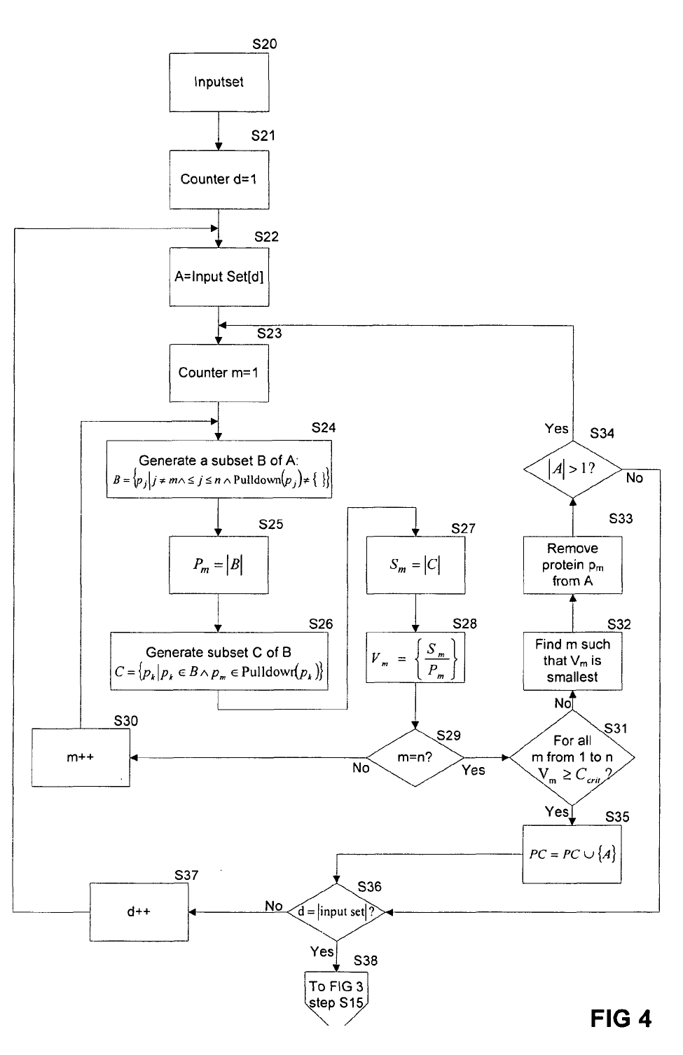

FIG. 4 is a flowchart showing part of the process of FIG. 3 in further detail;

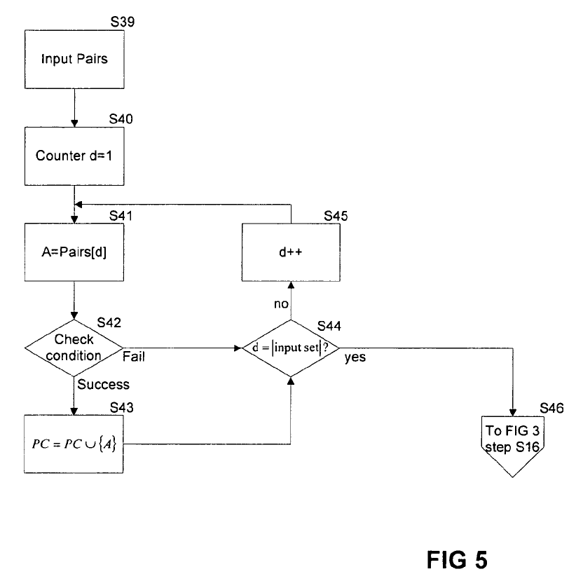

FIG. 5 is a flowchart showing part of the process of FIG. 3 in further detail;

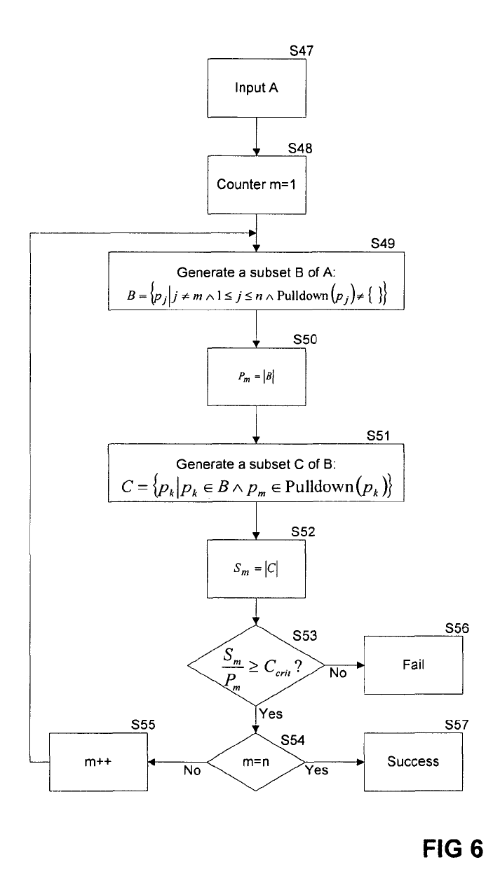

FIG. 6 is a flowchart showing part of the process of FIG. 5 in further detail;

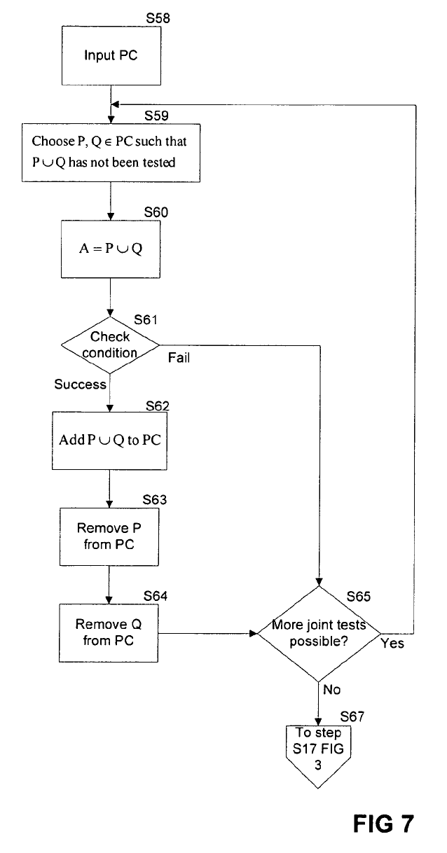

FIGS. 7 to 9 are flowcharts showing parts of the processing of FIG. 3 in further detail;

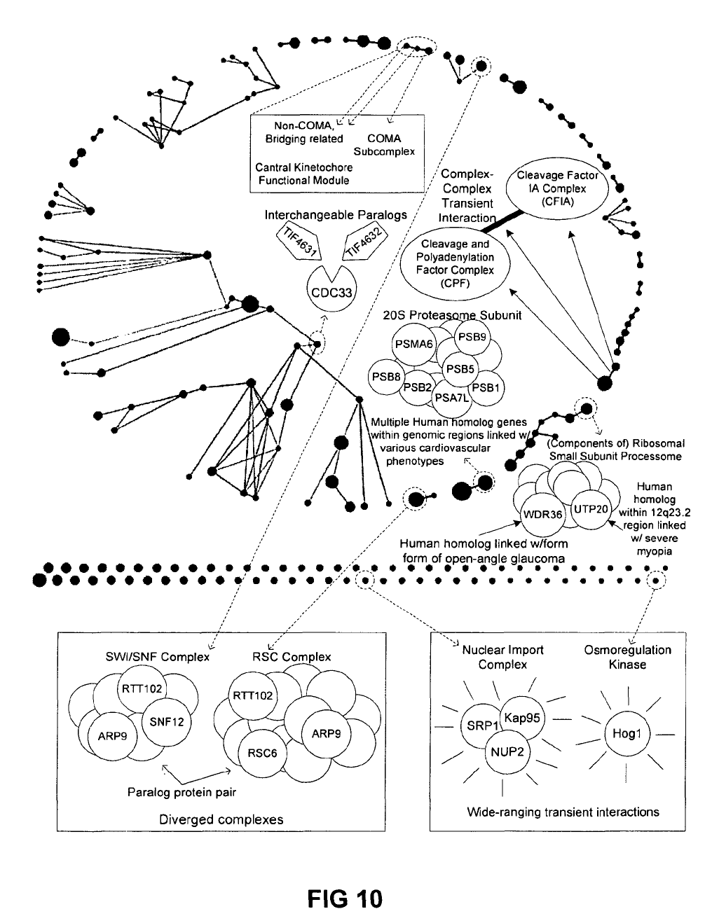

FIG. 10 is a schematic illustration of the S cerevisiae interactome;

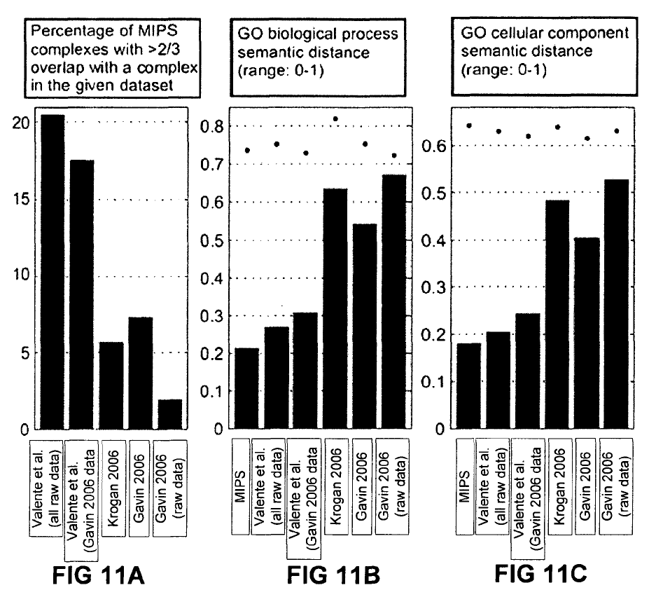

FIGS. 11A to 11C are graphs indicating the reliability of complexes predicted using the methods described herein;

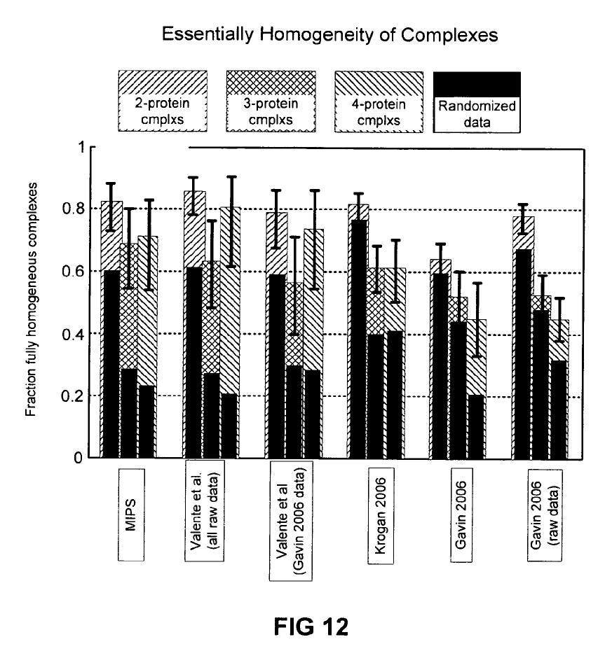

FIG. 12 is a graph indicating fractions of identified complexes that are fully homogeneous; and

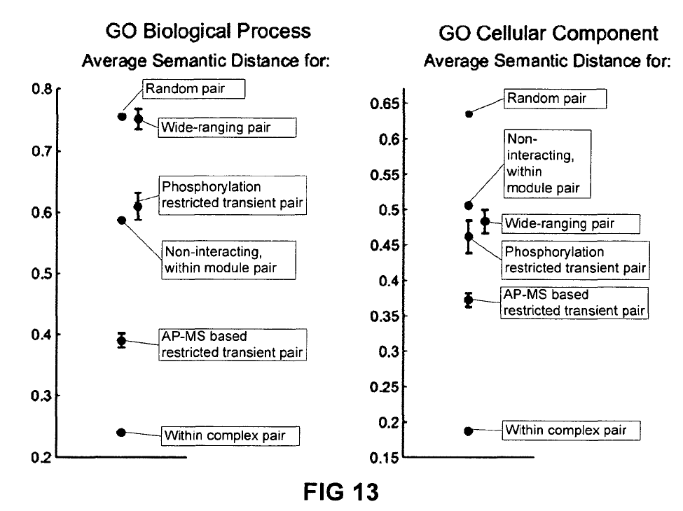

FIG. 13 is a schematic illustration showing average Semantic Distance for pairs of proteins in different interaction classes.

Referring to FIG. 1, the described embodiment takes as input a set of pull-down assay data 1 of the form:

a→{a,b,c,d} (1)

Where equation (1) indicates that protein a as a bait pulled down proteins a, b, c and d.

The embodiment further takes as input a set of proteins 2. The set of proteins 2 together with the pull down assay data 1 is input to an algorithm 3 which, as described below, generates a plurality of sets of proteins 4, each set of proteins being a permanent protein complex.

FIG. 2 shows the criterion which the algorithm 3 applies to determine whether a particular sub-set of proteins taken from the set of proteins 2 is a permanent protein complex. At step S1 a subset A of size n of the set of proteins 2 is generated, and is represented by equation (2):

A={pᵢ|1≦i≦n} (2)

A counter variable m is initialised to a value of 1 at step S2. The counter variable m will count through proteins in the set A. At step S3 a subset B of the set A is generated by selecting those proteins of the set A, other than the protein, indicated by the value of the counter variable m itself (pₘ) which are such that they pull down at least one other protein. That is, the subset B includes all proteins in the set A, other than the protein pₘ, which generate a non empty pull-down. The proteins pulled-down by a particular protein are determined with reference to the pull-down assay data 1. The set B is defined mathematically by equation (3):

B={pⱼ|j≠m [symbol] 1≦j≦n [symbol] Pulldown(pⱼ)≠{ }} (3)

where Pulldown (pⱼ) generates a set of proteins which are pulled-down by the use of pⱼ as a bait, as determined by the pull-down assay data 1.

At step S4 the cardinality of the set B is determined, and assigned to a variable Pₘ. It can thus be seen that the variable Pₘ indicates the number of proteins other than pₘ in the set A, which produce non-empty pull-downs.

At step S5 a subset C of the set B is generated. The set C contains proteins included in the set B which pull-down the protein pₘ as currently indicated by the counter variable m. The set C is defined by equation (4):

C={pₖ|pₖεB [symbol] pₘεPulldown(pₖ)} (4)

At step S6 the cardinality of the set C is assigned to a variable Sₘ. It can thus be seen that the variable Sₘ indicates the number of proteins in the set B which pull-down the protein pₘ.

At step S7 the value of a metric given by equation (5a) is determined and compared to a threshold C_{crit} as shown in equation (5b).

$$\begin{matrix}\frac{{S}_{m}}{{P}_{m}} = \{ \begin{matrix}0 if {P}_{m} = 0 \\ \frac{{S}_{m}}{{P}_{m}} otherwise\end{matrix} & ( 5 a ) \\ \frac{{S}_{m}}{{P}_{m}} \geq {C}_{crit} & ( 5 b )\end{matrix}$$

It can be seen from equation (5a) that the relationship is normally generated by straightforward division. However, if Pₘ is equal to 0, the division given by equation (5a) is not well defined given that it specifies division by zero. Therefore if Pₘ=0 the relationship of equation (5a) is defined to be zero and the inequality (5b) cannot be satisfied given that C_{crit} has a value greater than zero.

Excluding the case where Pₘ has a value of zero, given the definitions of Sₘ and Pₘ as described above it can be seen that equation (5a) specifies a required ratio of the number of pull-downs generated by proteins in the set of proteins A including the protein pₘ relative to the number of non-empty pull downs generated by proteins in the set of proteins A. The larger the value of the fraction included in equation (5) the stronger the relationship between the protein pₘ and other proteins included in the set A. It can be seen that pull-downs generated using pₘ itself as a bait are ignored for purposes of the ratio calculation specified by equation (5a).

In one embodiment of the invention the value of C_{crit} is 0.6. This was selected based upon evaluation of a range of possible values and the effect of these values on the reliability of the generated permanent complex data. It was found that variances in the value of C_{crit} of ±0.05 had only a small effect on the generated permanent complex data.

If the inequality of equation (5b) is not satisfied at step S7 it is determined that the set A is not a complex, on the basis that there is insufficient interaction between the protein pₘ and other proteins included in the set A. Processing therefore ends at step S8.

If the inequality of equation (5b) is satisfied, processing passes from step S7 to step S9 where a check is carried out to determine whether the counter variable m has a value of n. If this is the case, it can be determined that the processing described above has been carried out for each protein in the set A, and processing can continue at step S10 as described further below. If however the value of the counter variable m is not equal to n it can be determined that further proteins remain to be processed. In such a case processing passes from step S9 to step S11 where the value of the counter variable m is incremented, before processing returns to step S3.

When processing reaches step S10 it can be determined that there is sufficient relationship between all proteins in the set A for the set of proteins A to be one of the sets of proteins 4 output from the algorithm 2 as shown in FIG. 1. However at step S10 a check is required to determine whether the set A is in fact a subset of a larger set which when processed as described above would also result in processing reaching step S10. In such a case the set A is not defined as a complex, as it is the superset of the set A which is defined as the permanent protein complex.

For this reason, step S10 determines whether the set A is in fact a subset of a set which is it self a protein complex. If this is the case, the set A does not define a permanent protein complex and processing passes from step S10 to step S8. Otherwise, processing passes from step S10 to step S12 where it is recorded that the set A does define a permanent protein complex.

In some embodiments of the invention the set of pull-down assay data 1 (FIG. 1) may be generated from multiple datasets. As such a particular protein (referred to as pₚ) included in the set A may generate more than one non-empty pull down, each non-empty pull down being associated with one of the multiple datasets. In such a case the protein pₚ will have an undue influence on the values of Sₘ and Pₘ determined as described above. To avoid such a circumstance, while the value of Pₘ is determined as described above, the value of Sₘ is only increased by a fraction of the number of non-empty pull-downs generated by the protein pₚ which include pₘ to the total number of non-empty pull-downs generated by the protein pₚ. The contribution of pₚ to Pₘ, or equivalently to the cardinality of B in this case, is defined to still be 1. This allows the reliability of complex predictions to be improved by repeating experimental assays and combining datasets.

In preferred embodiments of the invention the values of Pₘ and Sₘ when calculated as described above are modified before being used by subtraction of a discount D. That is, equation (5b) is modified to be:

$$\frac{{S}_{m} - D}{{P}_{m} - D} \geq {C}_{crit} \qquad ( 5 c )$$

D is defined to be the largest integer which is such that the probability of obtaining by chance a value of Sₘ that is greater than or equal to D is equal to or larger than a predetermined threshold B_{crit}. The probability of obtaining a value of Sₘ that is greater than or equal to D by chance can be calculated using a basic randomization model that uses the net data ratio of equation (6) as the base probability that any given single assay pulls-down pₚ. For baits that had multiple assays in the dataset, a single assay is assumed in this random model.

$$\frac{No of proteins pulling down {p}_{m}}{No of proteins with a non \text{-} empty pull down} \qquad ( 6 )$$

It has been found that a value of B_{crit} of 0.01 works well in embodiments of the invention. This value was determined by evaluation of a range of possible values. Trials have shown that deviations of ±0.005 from the preferred value of B_{crit} have little effect on reliability.

By way of further explanation, the use of the variable D takes into account the number of proteins which pull down a particular protein pₘ. If the particular protein pₘ is pulled down by a large number of proteins, it can be seen from the preceding description that the value of D will be relatively large. Conversely if only a small number of proteins pull down the particular protein pₘ the value of D will be smaller. Thus, the value of D is proportional to the number of proteins pulling down the protein pₘ as compared with the number of proteins producing non-empty pull downs. That is, if a particular protein pₘ is pulled down by a large number of other proteins the fact that it is pulled down by a particular protein is considered to be less significant, and a larger value of D is therefore selected.

It can therefore be appreciated that the described method includes a statistical correction to account for proteins that tend to bind indiscriminately to other proteins, and/or to laboratory equipment (for example a purification column) used to derive the high throughput protein identification assay data, and therefore more easily fulfill the test by chance.

From the preceding description it can be seen that the determination of permanent protein complexes requires the determination of sets of proteins which satisfy the processing described with reference to FIG. 2. That is, the processing of FIG. 2 can be carried out for each of a plurality of sets A. It is very computationally expensive in terms of execution time to systematically apply the processing of FIG. 2 to all potential sets A of proteins in an organism or cell, indeed such processing is often practically impossible. This is particularly so given the large number of protein species typically in question. The inventors have therefore developed a method which identifies permanent protein complexes which allows complexes to be identified using widely available computing power. This method is now described.

The method for identifying permanent protein complexes is first described in overview with reference to FIG. 3.

At step S13 a set of potential complexes PC is initialised to be the empty set. At step S14 each data item included in the pull down assay data 1 is processed to determine whether it should be added to the set of potential complexes PC, as described in further detail below. At step S15 pairs of proteins are processed as described in further detail below to determine whether these pairs represent permanent protein complexes. At step S16 potential protein complexes in the set PC are merged to determine whether any merged complexes are themselves complexes, and again this processing is described in further detail below. At step S17 each potential permanent complex in the set PC is processed in turn by adding a single protein to the complex before carrying out further processing to determine whether the permanent complex with the addition of the single protein is itself a potential complex. The processing of steps S16 and S17 is repeated through the action of a loop at S18. At step S19 a coalescence process is carried out, and this process is again described in further detail below.

The processing of step S14 is now described in further detail with reference to FIG. 4. At step S20 the process takes as input the set InputSet which is a set of sets of proteins. The set InputSet is constructed by adding for each data item included in the pull down assay data of the form in equation (1), the sets of proteins pulled down by the particular baits. For example for the pull down assay data entry in equation (1), the set {a,b,c,d} is added to the set InputSet.

At step S21 a counter variable d is initialised to 1. At step S22 the set A is initialised to the d^{th} element of the set InputSet. Steps S23 to S27 can be seen to correspond to steps S2 to S6 of FIG. 2 and are therefore not described further here.

At step S28 the m^{th} element of a set V is provided with the value of the ratio shown in equation (7):

$$\frac{{S}_{m}}{{P}_{m}} = \{ \begin{matrix}0 if {P}_{m} = 0 \\ \frac{{S}_{m}}{{P}_{m}} otherwise\end{matrix} \qquad ( 7 )$$

Each element m of the set V indicates a strength of the relationship between a protein pₘ and other proteins included in the set A.

At step S29 the counter variable m is compared to the variable n corresponding to the size of the set A. If the values of m and n are equal, it can be determined that the processing described above has been carried out for each protein in the set A, and processing can continue at step S31 as described further below. If however the value of the counter variable m is not equal to n it can be determined that further proteins remain to be processed. In such a case processing passes from step S29 to step S30 where the counter variable m is incremented and the processing beginning at step S24 is repeated.

At step S31 each value in the set V is compared to the threshold C_{crit}. If each entry in the set V is larger than the threshold then it is determined that the set A is a potential complex and at step S35 the set A is added to the set of potential complexes PC and processing proceeds to step S36 as described below. If the check of step S31 is not satisfied processing passes to step S32.

At step S32 the smallest value in the set V is found. At step S33 the corresponding protein pₘ is removed from the set A. The size of the set A is determined at step S34 and if it is not greater than 1 the processing proceeds to step S36 as described below. If the size of the set A is greater than 1, the processing beginning at step S23 is repeated by the action of a loop, with the set A after the modification carried out at step S33 as input.

At step S36 the counter variable d is compared to the size of the set InputSet. If d is equal to the size of the set InputSet it is determined that each entry in the set InputSet has been tested and processing passes to step S15 (FIG. 3). If d is not equal to the size of the set InputSet, it is determined that further sets remain to be tested. At step S37 the value of d is incremented and the processing beginning at step S22 is repeated by the action of a loop.

The processing of step S15 of FIG. 3 is now described with reference to FIG. 5.

The processing shown in FIG. 5 takes as input at step S39 a set Pairs. The set Pairs is a set of all possible combinations of two proteins from the set of proteins. At step S40 a counter variable d is initialised to a value of 1. At step S41 the set A is initialised to be the d^{th} set of the set Pairs.

Step S42 of FIG. 5 comprises the plurality of sub-steps as shown in FIG. 6 and described in further detail below.

If the processing of step S42 returns “Fail” then processing passes from step S42 to step S44 as described below. If the processing of step S42 returns “Success”, then processing passes to step S43 where the pair A is identified as a potential complex and added to the set of potential complexes PC. Processing then proceeds to step S44.

At step S44 the counter variable d is compared to the size of the input set Pairs. If d is equal to the size of the set Pairs then no more pairs remain to be tested and processing passes to step S16 of FIG. 3. If d is smaller than the size of the input set then more pairs remain to be tested. Processing therefore passes to step S45 where the counter variable d is incremented and the processing beginning at step S41 is repeated by the action of a loop.

The processing shown in FIG. 6 which is carried out at step S42 of FIG. 5 is now described in further detail.

The processing takes as input at step S47 a set of proteins A. It can be seen that steps S48 to S55 correspond to the loop defined by steps S2 to S7, S9 and S11 of FIG. 2.

At step S53 if the inequality of equation (5) is not satisfied the process of FIG. 6 returns “Fail” to indicate that the set A is not a complex.

At step S54 if the counter variable m is equal to the counter variable n that defines the size of the set A, then at step S57 the process of FIG. 6 returns “Success” to indicate that the set A satisfies the required criteria.

The processing of step S16 of FIG. 3 is now described in further detail with reference to FIG. 7.

The processing of FIG. 7 takes as input at step S58 the set of potential complexes PC. At step S59 two potential complexes P and Q that have not been previously chosen for joint testing are selected from the set PC. At step S60 a set A is defined as the set union of P and Q.

Step S61 of FIG. 7 comprises the plurality of sub-steps shown in FIG. 6 and described above.

If step S61 returns “Success” then the set A is identified as a potential complex and is added to the set of potential complexes PC at step S62. At steps S63 and S64 the potential complexes P and Q are removed from the set of potential complexes PC given that their union is now treated as a complex. Processing then proceeds to step S65 which is described below. If step S61 returns “Fail” then the set A is not a potential complex and processing proceeds to step S65.

At step S65 either the set A has been identified as a potential complex and the set PC updated or the set A is not a potential complex and the set PC remains unchanged. In both cases step S65 identifies whether more pairs of potential complexes P and Q from PC remain to be jointly tested at step S59. If more tests are possible the processing of steps S59 to S65 is repeated through the action of a loop. If no new tests are possible processing passes to step S17 (FIG. 3). When a new potential complex is added to the set PC via a merge, this typically creates new possible tests as unions involving this new complex have not previously been tested.

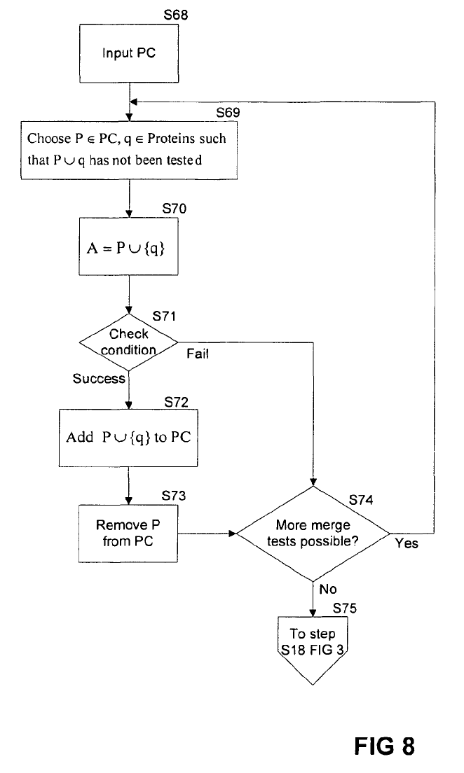

The processing of step S17 is now described in further detail with reference to FIG. 8.

The processing shown in FIG. 8 takes as input at step S68 the set PC of potential complexes and the set of proteins 2. At step S69 a potential complex P from the set PC and a single protein q from the set of proteins that is not in P are chosen for testing at step S69 subject to the potential complex P∪{q} not having been previously chosen for testing. At step S70 the set A is defined as the set union of P and q.

Step S71 of FIG. 8 comprises the plurality of sub-steps shown in FIG. 6 and described above.

If step S71 returns “success” then the set A is identified as a potential complex and is added to the set of potential complexes PC at step S72. At step S73 the potential complex P is removed from the set of potential complexes PC given that P∪{q} is now treated as a complex. Processing then proceeds to step S74 which is described below. If step S71 returns “fail” then the set A is not a potential complex and processing proceeds to step S74.

At step S74 either the set A has been identified as a potential complex and the set PC updated or the set A is not a potential complex and the set PC remains unchanged. In both cases step S74 identifies whether further tests are possible between single individual proteins in the set of proteins and potential complexes in the set PC. More tests are possible if there remain complexes in PC and proteins in the set of proteins that have not been jointly tested. If more tests are possible the processing of steps S69 to S74 is repeated through the action of a loop. If no more tests are possible processing passes to step S18 (FIG. 3). It should be noted that when a new potential complex is added to the set PC by processing described with reference to FIG. 8 this typically creates further possible complex-protein merges to which the processing of FIG. 8 can be applied, and these are handled by the loop of step S74.

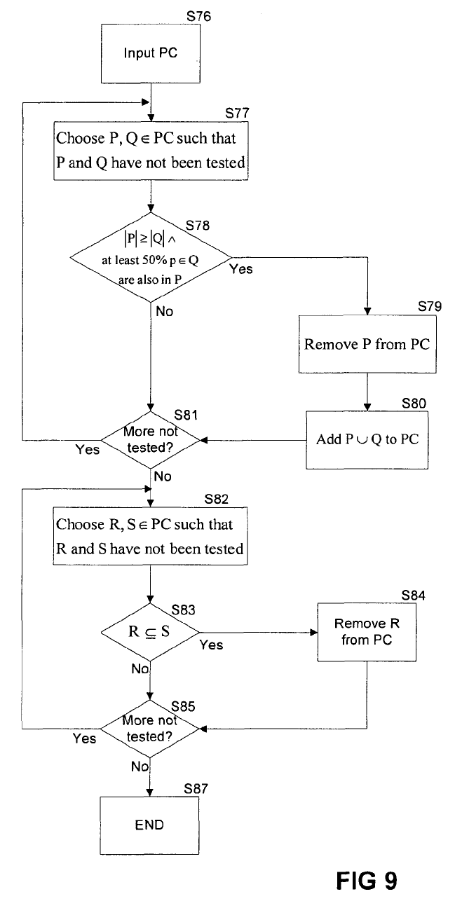

The processing of step S19 is now described in further detail with reference to FIG. 9.

The processing of FIG. 9 takes as input the set PC of potential complexes at step S76. At step S77 two potential complexes P and Q are chosen from PC such that P and Q have not previously been tested according to the test at step S78 described below.

At step S78 a check is carried out to determine whether the cardinality of P is greater than or equal to that of Q and at least fifty percent of the proteins pᵢ in Q are also in P. It will be appreciated that the fifty percent threshold is a value chosen from experimental data and other values may be suitable.

If the criterion of step S78 is satisfied then P is removed from the set PC at step S79 and at step S80 the proteins in Q are added to the proteins in P and this new potential complex is added to PC. The process then proceeds to step S81 described below. Note that this addition is made regardless of satisfaction of the criterion described in FIG. 6. If the criterion of step S78 is not satisfied processing proceeds directly to step S81.

At step S81 it is determined whether further potential complexes in PC have not been tested according to the condition at step S78. If this is the case the processing of steps S77 to S81 are repeated through the action of a loop. If there are no more potential complexes remaining that have not been tested according to the condition at step S78 processing passes from step S81 to step S82.

At step S82 two potential complexes R and S are chosen from PC such that R and S have not been tested according to the test at step S83 described below. At step S83 it is determined if R is a subset of S. If this is not the case then the process continues to step S85 described below. If R is a subset of S, at step S84 R is removed from the set PC and the process continues to step S85.

At step S85 it is determined if further potential complexes in PC have not been tested according to the subset condition at step S83. If further potential complexes have not been tested then steps S82 to S85 are repeated through the action of a loop. If further tests are not possible then the processing terminates.

It will be appreciated that processing as described above with reference to FIG. 3 allows a set of potential protein complexes to be generated. That is, the processing described with reference to FIG. 3 implements the algorithm 3 of FIG. 1. The set of potential protein complexes for output by the algorithm 3 is considered to be a set of permanent protein complexes. Further processing can then be carried out to identify transient interactions. Specifically, if the pull-down assay data generated by a particular protein p, where p is a member of a permanent protein complex P₁ but not a member of a permanent protein complex P₂, contains strictly more than 50% of the proteins of the permanent protein complex P₁ and strictly more than 50% of the proteins contained in the permanent protein complex P₂, then the permanent protein complexes P₁ and P₂ are defined to transiently interact.

Transient interactions of the type described above can be identified by checking every data item in the set of pull-down assay data 1 and every pair of permanent protein complexes included in the complexes for output by the algorithm 3 for satisfaction of the criterion set out above.

All of the features described herein (including any accompanying claims, abstract and drawings), and/or all of the steps of any method or process so disclosed, may be combined with any of the above aspects in any combination, except combinations where at least some of such features and/or steps are mutually exclusive.

Data generated using the methods described above will now be further described with reference to the following Example. The efficacy of the methods is also discussed with references to various comparisons performed between data generated using the methods described above and reference data.

### EXAMPLE 1

### Functional Organization of the Yeast Proteome By a Novel Yeast Interactome Map

### Introduction

The methods described above allow an interactome to be modeled in terms of i) predicted permanent (i.e. high-affinity) protein complexes and ii) predicted specific transient (i.e. lower affinity) interactions between such complexes and/or individual proteins, while discarding iii) generic, predicted less specific transient interactions. This falls in-between a detailed structural characterization of each interaction [10], and a binary protein-protein pairwise-only reporting of interactions [1, 2]. The former of these two, the arguable system's level functional relevance of the detail it provides aside, would certainly be hard to realize accurately in a large-scale fashion, due to current experimental limitations. The latter of the two, due to its scalability, can be very useful as a first approximation, but is ultimately less than ideal, as proteins do not work in a strict pairwise fashion [11], besides the fact that significant functional information can be lost under a purely on/off description of an interaction.

The methods described above to generate data usable to construct an interactome were developed based upon raw data from high-throughput affinity purification followed by mass spectrometric (AP-MS) identification assays [12, 13, 14]. A key premise used is that, under ideal conditions, every protein member of a given complex when used as a bait should pull-down every other protein in that same complex. Although this ideal is not attainable in practice due to a variety of experimental limitations, how close it comes to being fulfilled provides a measure of the certainty that a given group of proteins constitutes a complex in the cell.

In the light of the above observations, the problem becomes one of searching for sets of proteins that fulfill the above test to a specified minimum degree. As indicated above, the described methods include appropriate statistical corrections to account for proteins that tend to bind indiscriminately to other proteins and/or to the purification column itself, and which as such could more easily fulfill the test by chance.

### Results and Discussion

The methods described herein are ideally suited for large-scale AP-MS interactome mapping projects, as the reliability (both sensitivity and specificity wise) of its predicted complexes improves as the number of AP-MS assays performed increases (as described above). Taking raw data from three large-scale AP-MS studies on S cerevisiae [12, 13, 14], the methodology was applied to build an S cerevisiae interactome as described further below. Before excluding wide-ranging interactions as described above, the set of predicted transient interactions was enriched with kinase-substrate literature curated interactions [17]. The final interactome consists of 248 nodes (210 predicted multiprotein complexes and 38 single kinases) and 113 restricted transient interactions (65 predicted using the methods described herein and 48 phosphorylation literature interactions).

FIG. 10 shows the S cerevisiae interactome generated using the methods described herein. Circles referred to as nodes represent 210 predicted multiprotein complexes and 38 kinases (where node sizes proportional to complex sizes). Links between the circles represent 113 putative predicted restricted transient interactions between nodes (65 complex-complex predicted interactions and 48 kinase-substrate literature based interactions). The network is laid out in Polar Map fashion, with each topological module placed in a conical region, with some blank space in between the modules [7].

One complex and one kinase (HOG1) had more than the 8 cut-off number of predicted transient interactions, with those interactions being therefore classified as wide-ranging (as shown in FIG. 10). Subsequent examination showed this complex to be composed of three proteins, SRP1, KAP95 and NUP2, that are expected to transiently interact with many proteins/complexes in a function-nonspecific manner. These three proteins are all involved in nuclear protein import, and are known to interact with dozens of partners representing a broad range of functional categories [18, 19]. This is exactly the sort of wide-ranging interaction that it was wished to eliminate, one representing a standard function performed on many targets/complexes and that could occlude the role of more restricted interactions. Similarly, the protein kinase HOG1 is involved in a multitude of distinct cellular processes, including water homeostasis [20], arsenite detoxification [21], copper-resistance [22], hydrogen peroxide response [23], adaptation to citric acid stress [24], amongst others.

The quality of the interactome map was assessed via a number of distinct tests. First a set of 199 manually curated complexes from the MIPS database [25] (in a form further refined for accurateness by Lichtenberg et al. [26]) was used as a gold standard for comparison, including 199 complexes. FIG. 11A is a graph showing a Percentage of MIPS complexes with a greater than two-thirds overlap with a complex in a given dataset. The refined MIPS data is shown compared to:
data generated using the methods described above, based on combined raw AP-MS data from [12, 13, 14] (210 complexes) denoted “Valente et al (all raw data)”;
data generated using the methods described above based on APMS Gavin 2006 [13] raw data only (165 complexes) denoted “Valente et al (Gavin 2006 data)”;
complexes predicted by Krogan in [14] (546 complexes) denoted “Krogan 2006”;
complexes predicted by Gavin in [13] (491 complexes) denoted “Gavin 2006”; and
complexes predicted in the raw data of Gavin 2006—taking each raw pull down in [13] as a predicted complex, without computational treatment (1751 complexes) denoted “Gavin 2006 (Raw data)”.

The same data sets form the basis for FIGS. 11B and 11C described further below.

Secondly, in order to compare the reliability of protein complexes predicted using the methods described herein to that of the MIPS gold-standard itself, a non gold-standard based measure, termed Semantic Distance [27] was used. Semantic Distance (range: 0 to 1) provides an automated measure of the distance amongst a complex's protein members annotation-wise, in this case, based on the GO database Biological Process and Cellular Component annotations [28, 29]. This is shown in the graphs of FIGS. 11B and 11C where dots represent results under randomization of the respective datasets (standard deviation values smaller than dot size). These tests showed that the average semantic distance amongst proteins within each of the complexes predicted using the methods described herein comes close to that for the gold-standard MIPS complexes. Further, it is relevant to note that some of the GO database protein annotations and some of the MIPS dataset complexes may be based on the same literature source, artificially deflating, to an undetermined extent, the semantic distance within MIPS complexes. Seemingly, this should be most pronounced in the case of the Biological Process annotation.

A complex is defined to be essentiality-wise fully homogeneous if either i) knock-out of any one of its member proteins is lethal to the cell or ii) no single member protein knock-out is lethal. The fraction of essentiality-wise fully homogeneous complexes in a dataset as is presented as a third quality test [30, 31, 32] and is shown in FIG. 12. Analysis was performed separately for complexes of sizes 2, 3 and 4 to avoid size related biases (no statistically significant data for larger sized complexes was available). Error bars show 90% confidence interval for the underlying homogeneity fraction. Solid grey ‘randomized data’ bars show expected homogeneity fraction under randomization of the respective data (see methods). Dataset source references are as noted above with reference to FIG. 11A, and like annotations have been used. A major advantage of this third quality test is the apparent lack of significant hidden biases or sources of noise: the essentiality classification for most yeast proteins is reliable and the test involves neither the use of a less than perfect gold-standard nor comparisons based on annotations that are always subjective by nature. In this sense, the error bars shown in FIG. 12 likely constitute a correct, non-underestimated, assessment of the error associated with the test; an error which will decrease, as the net number of predicted complexes increases in future studies. In this study, it is already worth noticing how the homogeneity above random (difference between the background patterned bars and the respective foreground solid grey bars) of the complexes predicted using the methods described herein is comparable to that of the MIPS complexes, for both 2-protein, 3-protein and 4-protein sized complexes. Taken together with the semantic distance results, this leads the inventors to conclude that the integration of the methods described above with the latest AP-MS high-throughput experimental techniques [13, 14] allows large-scale prediction of complexes with a reliability typical of low-throughput experiments.

As noted above, having built a set of permanent complexes, further information was extracted from the AP-MS raw data by building a set of predicted putative transient interactions between the permanent complexes, as shown in FIG. 10. Being of lower affinity, such interactions are naturally harder to discern, present day literature data on transient complex-complex interactions being itself still comparatively sparse. This precludes a better net assessment of the reliability of the transient interaction predictions. Given also the lower stringency of this algorithm (vis-à-vis the complex prediction algorithm), the greater uncertainty over the reliability of these predictions should be emphasized. Nonetheless, Semantic Distance tests show that for both the GO Biological Process and the GO Cellular Component annotations, the average Semantic Distance associated with this class of interactions is higher than the respective average for permanent complexes, while lower than the respective average for the class of wide-ranging interactions shown in FIG. 13, consistent with expectations.

Values shown in FIG. 13 were calculated as follows. Within complex pair—average Semantic Distance over all pairs of proteins A and B, where A and B are found in the same predicted permanent complex. AP-MS based predicted restricted transient interaction pair—average Semantic Distance over all pairs of distinct proteins A and B, where A and B are in distinct predicted complexes that interact via an AP-MS data based predicted transient restricted interaction. Phosphorylation restricted transient interaction pair—as in the previous case, but where the restricted transient interaction is now based on a kinase-substrate literature reported interaction. Wide-Ranging pair—average Semantic Distance over all pairs of distinct proteins A and B, where A and B are in distinct predicted complexes that interact via a transient interaction (either predicted or kinase-substrate literature based) classified as wide-ranging. Non-interacting, within module pair—average Semantic Distance over all pairs of distinct proteins that belong to the same topological module but that do not fall within any of the cases above. Random pair—average Semantic Distance over all pairs of proteins present in the dataset. Assuming independence of the observed Semantic Distances for pairs in a given class, 95% confidence intervals for the predicted averages are shown (unless confidence interval is smaller than data point size). The presence of correlations means these are underestimates of the true, hard to quantify, errors (see Methods). X-axis placement of data points chosen just for clarity.

As a concrete example, the methods described herein predicted a complex mainly comprised of protein components of the cleavage and polyadenylation factor complex (CPF) to transiently interact with a complex mainly comprised of protein components of the cleavage factor IA complex (CFIA) (shown in FIG. 10). The CPF and CFIA complexes are both involved in the process of transcript poly(A) tail synthesis and maturation and are known to transiently interact as part of this process (see, for instance, Mangus et al. [33]).

In the past, S cerevisiae underwent a whole-genome duplication event [34]. A total of 22 paralog protein pairs originating at this single event fall within the interactome created using the methods described herein. In only 1 of these 22 pairs, do the two proteins appear in distinct complexes. This happens to also be the pair furthest apart in terms of protein sequence homology (as per Blastp [35] score). From the other 21 within complex paralog pairs, 18 are viable-viable pairs (i.e., single knock-out of either of the paralogs is viable), with the remaining 3 being viable-lethal pairs (i.e., one of the paralogs is essential). Genetic interactions [36, 37] are reported in the SGD database [29] for 12 of the viable-viable pairs and for 1 of the viable-lethal pairs (a dosage rescue case of SEC24 by SFB2 [38]). Note that the absence of reported genetic interactions for the other cases could be simply due to lack of testing. Altogether, this evidence points to a picture where two paralogs could remain similar enough to be redundant and used interchangeably in a complex (19 potential such cases); paralogs could evolve to having non-interchangeable roles, as evidenced by possession of distinct knock-out phenotypes (with no known dosage rescue interaction), but still work within the same complex, as a reminiscence of their common evolutionary origin (2 potential such cases); paralogs could diverge to the point of acquiring roles within different complexes altogether (1 potential such case). This observed latter case, may conceivably illustrate the eventual functional divergence of a complex into two complexes with separate but still closely related functions: The two paralogs, SNF12 and RSC6, are found in two different complexes that, although distinct, are functionally related and share a subset of proteins in common [18] (FIG. 10). SNF12 is a component of the SWI/SNF complex, and RSC6 is a component of the chromatin structure remodeling complex (RSC). Both of these complexes promote ATP-dependent remodeling of chromatin and thus serve to regulate gene expression [39]. In contrast, the paralogs TIF4631 and TIF4632 may exemplify the prior case of paralogs that can be interchangeably used within a complex (FIG. 10). Both are individually nonessential, but together they form a synthetic lethal pair. They are predicted to be part of a complex whose remaining member, CDC33, is essential (FIG. 10). This opens the possibility that the complex is performing some critical role within the cell and that its functionality requires both CDC33 and either one of the two paralogs.

The full homogeneity essentiality-wise of many of the permanent complexes (FIG. 12) hints that this property is oftentimes intrinsic to the complex and to its role, rather than to its individual proteins. Likewise, certain pathologies may be more correctly assigned to an intrinsic malfunction of a complex as a whole, rather than to an individual or loose set of proteins [40, 41, 42]. With this in mind, the constructed yeast interactome was lifted to human via homology [43] and checked how known disease associated genes and chromosomal loci relate to the constructed interactome map. Interestingly, a number of cases, potentially 8, pointing in this direction were found. An example of related phenotypes mapping to the same complex is provided by a complex containing the gene PSMA6 (FIG. 10). A specific variant of this gene is known to confer susceptibility to myocardial infarction in the Japanese population [48]. A linkage to a related phenotype, susceptibility to premature myocardial infarction, has been reported at 1p36-34 [49] (again, no causative gene has yet been identified). This region includes PSMB2, another gene in the same complex. Linkage between various other cardiovascular phenotypes and genomic regions including genes from this complex have also been reported, e.g., linkage between familial atrial septal defect and 6p21.3 [50], a region that includes PSMB8 and PSMB9, genes that are also present in the complex.

There is by now accumulated evidence that protein complexes define a distinct, relevant scale of functional organization in the cell [12, 13, 14, 11]. Perhaps a subsequent higher-level scale of functional organization is provided by functional modules, or pathways, involving groups of complexes/proteins that transiently interact. As an attempt to probe for such hypothetical organization, the interactome is divided into topological modules that are dense in predicted restricted transient interactions (FIG. 10) [7, 51]. Individually, the functional relevance of some modules is immediately apparent. For instance, one module consists of three complexes whose proteins are all clearly related: each is a subunit of the central kinetochore, mediating the attachment of the centromere to the mitotic spindle. One of the complexes appears to be mainly comprised of proteins from the COMA subcomplex, a group of proteins that together bridge subunits in direct contact with DNA to those bound to microtubules [52]. The other two complexes are also comprised of proteins with a similar bridging function, but these proteins are not members of the COMA subcomplex [53]. With this modular breakdown, the predicted interactome has been organized in terms of i) permanent complexes, restricted ii) AP-MS based transient interactions and iii) phosphorylation transient interactions, iv) topological modules based on restricted transient interactions and v) wide ranging transient interactions. Of note are the Biological Process distinct average Semantic Distances for these classes (FIG. 13), overall supporting this proposed structuring of the interactome. By comparison, regarding Cellular Component average Semantic Distances (FIG. 13), wide-ranging interactions are now comparable to phosphorylation restricted transient interactions, with even AP-MS based restricted transient interactions being now closer to both of these than to permanent complexes, unlike they were Biological Process wise. This is consistent with the more homogeneous nature physical-location-wise of all transient interactions, the distinction amongst these classes being fundamentally a functional one (in the sense defined by the Biological Process GO annotation). Another observed difference, is the now slightly higher average Semantic Distance for modules than for all transient interaction types, even wide-ranging ones, which is consistent with modules being more physically extended over multiple cellular components. Nonetheless, combining the uncertainty in the different classes' average Semantic Distances (see FIG. 13) with the incompleteness and degree of inherent subjectivity of the GO annotations, collection of further data will be necessary to confirm the biological relevance of some of the interaction classes that have been put forward.

As mentioned above To the 65 AP-MS based predicted complex-complex transient interactions, 48 kinase-substrate restricted transient interactions curated from the literature [17] were added (an additional 9 interactions involving the HOG kinase were classified as wide-ranging). For kinase or substrate proteins that were members of one of the predicted complexes, the transient interaction was taken to involve the respective complex. Note that an additional 81 kinase-substrate literature curated interactions present in the same database [17] were not used in this work as they did not involve any protein present in the 210 predicted complexes dataset.

It was described that the overlap of generated complexes with MIPS complexes was considered, and this is shown in FIG. 11A as described above. Given two complexes, their fractional overlap is defined as:

$$overlap = \frac{No of protein species common to both complexes}{Net No of protein species in the two complexes} \qquad ( 8 )$$

For example, if:
complex A={a, b, c} and
complex B={b,c,d},
   then their overlap is

$$\frac{2}{4} = \frac{1}{2}$$

In the Gavin 2006 raw dataset, only pull-downs where at least one protein other than the bait was identified were considered.

It was also described above that to determine the semantic distance between two genes (or respective proteins) the method of Lord et al. [27] was used, except that ‘is-a’ and ‘part-of’ edges were treated equivalently. Briefly, the semantic distance between two GO terms in a given aspect, e.g., biological process, depends on the frequency of usage of the ‘minimal subsuming parent term’, i.e., the least commonly occurring GO term that is a parent term of both GO terms being compared. A GO term has ‘occurred’ when that term or any of its child terms is used in an annotation. So, for example, if the minimum subsuming parent term of two GO terms is the root, ‘biological process’, the GO terms being compared are far apart, since the frequency of the minimal subsumer is 1.0 (this term always occurs in an annotation, because any term in the biological process aspect is one of its children; even if no terms are assigned to a gene product, one can still assign the generic term ‘biological process’). On the other hand, if the frequency of the minimal subsumer is strictly less than 1.0, this implies that the GO terms being compared are highly similar since they are both part of the same, very specific (rarely used) subgraph. If the two terms being compared are in fact the same term, then the minimal subsumer is the term itself.

Specifically, the frequency of usage for any term is defined as:

p(termX)=number of times that term X occurs/number of times any term occurs.

The semantic distance between two terms, A and B, is then defined as [54]

$$SD = 1 - \frac{2 ln ( p ( minimal\_subsummer ) )}{ln ( p ( A ) ) + ln ( p ( B ) )}$$

If A=B, then p (A)=p (B)=p(minimal_subsumer) and SD=0. On the other hand, if the minimal subsumer of A and B is the root term, then p(minimal_subsumer)=1 and SD=1.

Because a gene may be annotated with more than one GO term for a given aspect, the semantic distance between genes P and Q is defined as the average of the pairwise term distances, one member of the pair from gene P and the other from gene Q. GO term frequency was calculated using the June, 2007 GO database [28], including all evidence codes. The Saccharomyces cerevisiae annotation file was downloaded from the GO website on Jul. 20, 2007 [29].

In the semantic distance values shown in FIGS. 11B and 11C, the following procedure was employed to ensure that differences on the typical complex size on different datasets did not lead to biases that would prevent a valid comparison amongst the different datasets average Semantic Distances.

The semantic distance of a complex is the average semantic distance of all the pair-wise combinations of protein members of that complex. The semantic distance of a dataset is calculated by:
1. Separately calculating the mean semantic distance for all complexes of each given size.
2. Averaging the different complex sizes average semantic distances.

It should however be noted that complexes containing any proteins without the relevant GO annotation were excluded from the respective semantic distance calculation.

Furthermore, semantic distances were calculated only for complexes of size up to and including 6, due to the statistically small number of complexes beyond this size.

A base random case semantic distance was calculated for each dataset (dots in FIGS. 11B and 11C). This was done by:
1. Randomizing the dataset via a large number of pairwise protein permutations amongst the complexes.
2. Calculating this randomized dataset semantic distance as described above.

It should be noted that standard deviations were determined for the randomized dataset semantic distances by repeating 50 times the above process for each dataset, and they were smaller than the data point size in FIGS. 11B and 11C.

The essentiality homogeneity of complexes in FIG. 12 was determined as follows. Patterned bars: For each dataset and complex size, the underlying Fraction of Fully Homogeneous Complexes from where the observed data was drawn is estimated in a Bayesian [55] fashion, assuming a prior probability uniform in the [0, 1] interval. The statistical mode (#fully homogeneous complexes observed/#total complexes observed) is reported in the main bar. The error interval reports the 90% confidence interval for this underlying fraction.

Solid grey bars: The expected homogeneity under randomization of the data (the foreground grey bar) is calculated based on the net fraction of lethal protein appearances (i.e., the same protein species appearing in two different complexes is counted twice for purposes of calculating this lethal fraction) on complexes of the size in question, for the given dataset. For example, for complexes of size 3, if 0.4 of the protein appearances in complexes of size 3 in the dataset are essential proteins and 0.6 are non-essential then it is expected for 0.4³+0.6³=0.28 of the complexes to be fully homogeneous essentiality-wise (since the complex could be “fully homogeneous lethal” or “fully homogeneous viable”).

Throughout, complexes where it was not known the essentiality of every member protein were excluded from the analysis. No statistically significant data was available for complexes of sizes larger than those reported.

In the case of semantic distance data as shown in FIG. 13, the confidence interval for the average Semantic Distance is calculated by assuming a Gaussian distribution for its predictor X (via the Central Limit Theorem), hence leading to a 95% confidence interval of the form

$$( X - 1.96 \frac{σ}{\sqrt{n}} , X + 1.96 \frac{σ}{\sqrt{n}} )$$

where n is the number of pairs tested and σ is approximated by the observed sample standard deviation. This confidence interval estimate assumes independence of the observed pair Semantic Distances in a given interaction class. However, in reality correlations of multiple kinds are present (e.g. the Semantic Distances for the pairs of proteins (A, B) and (A, C) are not independent in general, due to having protein A in common). This makes the error bars in FIG. 13 underestimate the true, hard to quantify, errors.

A homologous human version of the yeast interactome was obtained by matching each yeast protein to its human inparalog proteins, as per the Inparanoid database [43].

The ‘Q-modularity’ algorithm of Newman [7, 51] was applied to clustering the network of transient interactions. In this algorithm, the basic criterion for selecting the partition into modules is that the fraction of within-module transient interactions is maximized with respect to a base random case.

### REFERENCES

[1] Rual J-F, of al. (2005) Nature 437: 1173-1178.
[2] Stelzl U et al. (2005) Cell 122 (6): 957-68.
[3] Ewing R M et al (2007) Mol Sys Bio 3: 89.
[4] Lim J of al. (2006) Cell 125 (4):645-647.
[5] Ahn A C, TewariM, Poon C-S, Phillips R S. (2006) PLOS Medicine 3 (6): e208.
[6] Ahn A C, Tewari M, Poon C-S, Phillips R S. (2006) PLOS Medicine 3 (7): e209.
[7] Valente AXCN, Cusick M E. (2006) Nucleic Acids Research 34 (9): 2812-2819.
[8] Cusick M E, Klitgord N, Vidal M, Hill D E. (2005) Hman Molecular Genetics 14: R171-R181.
[9] Uetz P, Finley Jr. R L. (2005) Febs Letters 579: 1821-1827.
[10] Russel R B et al. (2004) Current Opinion in Structural Biology 14: 313324.
[11] Alberts B. (1998) Cell 92: 291-294.
[12] Gavin, A-C of al. (2002) Nature 415: 141-146.
[13] Gavin A-C et al. (2006) Nature 440 (7084):631-636.
[14] Krogan N J et al. (2006) Nature 440 (7084):637-643.
[15] Korcsm´aros T, Kov´acs I A, Szalay M S, Csermely P. (2007) J Biosci 32 (3): 441-446.
[16] Barab´asi A-L, Oltvai Z N. (2004) Nature Rev Genet 112: 101-114.
[17] Kinase and phosphatase database. (2007). http://www.proteinlounge/.
[18] Hertz-Fowler C et al. (2004) Nucleic Acids Res. 1; 32 (database issue): D339-43.
[19] Wente S R. (2000) Science 288 (5470): 1374-1377.
[20] Proft M, Struhl K. (2002) Mol.Cell. 9 (6): 1307-17.
[21] Sotelo J, Rodrguez-Gabriel M A. (2006) Eukaryot. Cell. 5 (10): 1826-30.
[22] Toh-e A, Oguchi T. (2001) Genes Genet. Syst. 76 (6): 393-410.
[23] Haghnazari E, Heyer W D. (2004) DNA Repair (Amst) 3 (7): 769-76.
[24] Lawrence C L, Botting C H, Antrobus R, Coote P J. (2004) Mol. Cell. Biol. 24 (8): 3307-23.
[25] Mewes H W, et al. (2002) Nucleic Acids Res 30: 31-34.
[26] Lichtenberg U, Jensen L J, Brunak S, Bork P. (2005) Science 307: 724-727.
[27] Lord P W, Stevens R D, Brass A and Goble C A. (2003) Bioinformatics 19: 1275-1283.
[28] Ashburner M et al. (2000) Nature Genetics 25 (1): 25-29.
[29] SGD project. (2007). “Saccharomyces Genome Database” http://www.yeastgenome.org/.
[30] Dezs\ddot{}“o Z, Oltvai Z N, Barab´asi A-L. (2003) Genome Research 13: 2450-2454.
[31] Winzeler E A et al. (1999) Science 285: 901-906.
[32] Giaever G et al. (2002) Nature 418: 387-391.
[33] Mangus D A, Smith M M, McSweeney J M, Jacobson A. (2004) Mol Cell Biol 24 (10): 4196-206.
[34] Kellis M, Birren B W, Lander E S. (2004) Nature 428 617-624.
[35] Altschul S F, Gish W, Miller W, Myers E W, Lipman D J. (1990) J. Mol. Biol. 215: 403-410.
[36] Boone C, Bussey H, Andrews B H. (2007) Nature Reviews Genetics 8: 437-449.
[37] Kelley R, Ideker T. (2005) Nature Biotechnology 23: 561-566.
[38] Higashio H, Kimata Y, Kiriyama T, Hirata A, Kohno K. (2000) J Bio Chem 275 (23): 17900-17908.
[39] Sengupta S M. (2001) J. Biol. Chem. 276 (16): 12636-12644.
[40] Kasper L et al. (2007) Nature Biotechnology 25: 309-316.
[41] Oti M, Snel M, Huynen M A, Brunner H G. (2006) Journal of Medical Genetics 43: 691-698.
[42] Chaudhuri A, Chant J. (2005) Bioessays 27: 958-969.
[43] O'Brien K P, Remm M, Sonnhammer E L L. (2005) Nucleic Acids Research 33: D476-D480.
[48] Ozaki K et al. (2006) Nat Genet. 38 (8): 921-5.
[49] Wang Q. (2004) Am. J. Hum. Genet. 74 (2): 262-271.
[50] Mohl W, Mayr W R. (1977) Tissue Antigens 10 (2): 121-2.
[51] Clauset A, Newman M E J, More, C. (2004) Physical Review E 70: art. no. 066111.
[52] De Wulf P, McAinsh A D, Sorger P K. (2003) Genes Dev. 17 (23): 2902-2921.
[53] Meraldi P, McAinsh A D, Rheinbay E, Sorger P K. (2006) Genome Biol. 7 (3): R23.
[54] Lin D. (1998). An Information-Theoretic Definition of Similarity. In Proceedings of the Fifteenth International Conference on Machine Learning, Morgan Kaufmann Publishers Inc. 296-304.
[55] Beaumont M A, Rannala B (2004) Nature Reviews Genetics 5: 251-261.

1. A method of generating data indicating whether a set of proteins is a protein complex, the method comprising:
receiving as input experimental data indicating experimentally observed relationships, each experimentally observed relationship being between a first protein and zero or more second proteins;
generating data indicating whether the set of proteins is a protein complex by processing said experimental data to determine:
a first data value indicating a number of proteins having a relationship with one or more second proteins; and
a second data value indicating a number of proteins having a relationship with a selected protein.

2. A method according to claim 1, wherein said generating comprises generating relationship data indicating a relationship between said first data value and said second data value, and said data indicating whether the set of proteins is a protein complex is based upon said relationship data.

3. A method according to claim 1, wherein said generating comprises generating relationship data indicating a relationship between said first data value and said second data value, and said data indicating whether the set of proteins is a protein complex is based upon said relationship data, and wherein said generating data indicating whether the set of proteins is a protein complex comprises determining whether said relationship data satisfies a predetermined condition.

4. A method according to claim 1, wherein said generating comprises generating relationship data indicating a relationship between said first data value and said second data value, and said data indicating whether the set of proteins is a protein complex is based upon said relationship data, wherein said generating data indicating whether the set of proteins is a protein complex comprises determining whether said relationship data satisfies a predetermined condition, and wherein said predetermined condition is defined with reference to a threshold.

5. A method according to claim 1, wherein said generating comprises generating relationship data indicating a relationship between said first data value and said second data value, and said data indicating whether the set of proteins is a protein complex is based upon said relationship data, and wherein said generating data indicating whether the set of proteins is a protein complex comprises determining whether said relationship data satisfies a predetermined condition
wherein generating data indicating whether the set of proteins is a protein complex comprises:
generating data indicating that the set of proteins is a protein complex if said predetermined condition is satisfied.

6. A method according to claim 1,
wherein said generating comprises generating relationship data indicating a relationship between said first data value and said second data value, and said data indicating whether the set of proteins is a protein complex is based upon said relationship data, and wherein said generating data indicating whether the set of proteins is a protein complex comprises determining whether said relationship data satisfies a predetermined condition
wherein generating data indicating whether the set of proteins is a protein complex comprises:
generating data indicating that the set of proteins is a protein complex if said predetermined condition is satisfied,
wherein the method comprises generating data indicating that the set of proteins is a protein complex if but only if the set of proteins is not a subset of another set of proteins which is a protein complex.

7. A method according to claim 1, wherein said generating comprises generating relationship data indicating a relationship between said first data value and said second data value, and said data indicating whether the set of proteins is a protein complex is based upon said relationship data, and wherein said generating data indicating whether the set of proteins is a protein complex comprises determining whether said relationship data satisfies a predetermined condition
wherein the method further comprises:
generating data indicating that the set of proteins is not a protein complex if said predetermined condition is not satisfied.

8. A method according to claim 1, further comprising:
storing data indicating the set of proteins;
wherein said first data value indicates a number of proteins in said set, other than said selected protein, having a relationship with one or more second proteins, and said second data value indicates a number of proteins in said set, having a relationship with the selected protein.

9. A method according to claim 1, further comprising:
storing data indicating the set of proteins;
wherein said first data value indicates a number of proteins in said set, other than said selected protein, having a relationship with one or more second proteins, and said second data value indicates a number of proteins in said set, having a relationship with the selected protein;
selecting each protein of the set of proteins in turn to be said selected protein;
generating a plurality of first data values, one for each protein of the set of proteins;
generating a plurality of second data values, one for each protein of the set of proteins.

10. A method according to claim 1, further comprising:
storing data indicating the set of proteins;
wherein said first data value indicates a number of proteins in said set, other than said selected protein, having a relationship with one or more second proteins, and said second data value indicates a number of proteins in said set, having a relationship with the selected protein;
selecting each protein of the set of proteins in turn to be said selected protein;
generating a plurality of first data values, one for each protein of the set of proteins;
generating a plurality of second data values, one for each protein of the set of proteins;
generating relationship data for each protein in the set of proteins based upon respective first and second data values, wherein said set of proteins is identified as a protein complex if but only if the relationship data for each protein in the set of proteins satisfies a predetermined condition.

11. A method according to claim 10, wherein said predetermined condition is defined with reference to a threshold.

12. A method according to claim 1, wherein said experimental data indicating experimentally observed relationships comprises a plurality of relationships between a particular first protein and a respective zero or more second proteins.

13. A method according to claim 1, wherein said experimental data indicating experimentally observed relationships comprises a plurality of relationships between a particular first protein and a respective zero or more second proteins, and wherein determining a number of proteins having a relationship with the selected protein comprises determining a proportion of the plurality of relationships indicating that the particular first protein has a relationship with the selected protein.

14. A method according to claim 1, further comprising modifying at least one of the first and second data values based upon a number of first proteins in the experimental data having a relationship with the selected protein.

15. A method according to claim 1, further comprising modifying at least one of the first and second data values based upon a number of first proteins in the experimental data having a relationship with the selected protein, wherein said modifying is further based upon a number of first proteins in the experimental data having a relationship with one or more other proteins.

16. A method according to claim 1, further comprising modifying at least one of the first and second data values based upon a number of first proteins in the experimental data having a relationship with the selected protein wherein said modifying the at least one of the first and second data values uses a discount value which is defined with reference to a probability of obtaining by chance a value of the second data value greater than or equal to said discount value.

17. A method according to claim 1, wherein said set of proteins is defined with reference to one or more second proteins with which a first protein has a relationship.

18. A method according to any preceding claim, wherein said experimental data is pulldown assay data.

19-36. (canceled)

37. A computer program comprising computer readable instructions controlling a computer to carry out a method according to claim 1.

38. (canceled)

39. Apparatus for generating data indicating whether a set of proteins is a protein complex, the apparatus comprising:
a memory storing processor readable instructions; and
a processor configured to read and execute instructions stored in said program memory;
wherein the processor readable instructions comprise instructions controlling the processor to carry out a method according to claim 1.
